(19) United States
(12) Patent Application Publication
(10) Pub. No.: US 2006/0275370 A1
(43) Pub. Date: Dec. 7, 2006

(54) Method and compositions for treatment of epithelial damage

(76) Inventors: Yih-Lin Chung, Taipei (TW); Nam-Mew Pui, Taipei City (TW)

Correspondence Address: FISH & RICHARDSON PC, P.O. BOX 1022, MINNEAPOLIS, MN, 55440-1022, US

(21) Appl. No.: 11/499,936

(22) Filed: Aug. 7, 2006

Related U.S. Application Data
(63) Continuation-in-part of application No. 10/843,025, filed on May 10, 2004.
Application No. 10/843,025 is a continuation-in-part of application No. 10/798,119, filed on Mar. 11, 2004.
Application No. 10/798,119 is a continuation-in-part of application No. 10/205,738, filed on Jul. 25, 2002, now Pat. No. 6,809,118.

(51) Int. Cl. A61K 9/14; A61K 9/127; A61K 38/12; A61K 31/19; A61K 31/56; A61K 31/198

(52) U.S. Cl. 424/486; 514/9; 514/575; 514/557; 514/565; 424/450; 514/171; 514/454

(57) ABSTRACT

The present invention is directed to methods and compositions of treating or preventing epithelial lining tissue damage from dermatitis or mucositis induced by radiation exposure and/or chemotherapy, by applying to skin, mucosa or other tissues of the body an amount of a therapeutic composition which comprises a histone deacetylase inhibitor formulated with at least one pharmaceutically acceptable biocompatible polymer or carrier, or pharmaceutically acceptable salts in an amount sufficient to delay onset or decrease severity of the signs and symptoms of dermatitis and mucositis in cancer therapy. Such therapeutic compositions have the advantage of prolonged retention and sustained action of the histone deacetylase inhibitor in the skin, mucosa or other tissues of the body. The invention is also directed to treatment and prevention of gastrointestinal distress and cancer-related fatigue syndrome that are associated with mucositis in cancer therapy.

### CROSS REFERENCE TO RELATED APPILCATIONS

This application is a Continuation-In-Part of pending U.S. patent application Ser. No. 10/843,025, filed May 10, 2004 and entitled “Histone Hyperacetylating Agents For Promoting Wound Healing and Preventing Scar Formation”, which is a Continuation-In-Part of pending U.S. patent application Ser. No. 10/798,119, filed Mar. 11, 2004 and entitled “Method for increasing therapeutic gain in radiotherapy and chemotherapy”, which is Continuation-In-Part of Ser. No. 110/205,738, filed Jul. 25, 2002 (now U.S. Pat. No. 6,809,118) and entitled “Methods for therapy of radiation cutaneous syndrome”.

### BACKGROUND OF THE INVENTION

1. Field of the Invention

The present invention relates to methods and compositions for treating or preventing epithelial lining tissue damage from dermatitis or mucositis induced by radiation exposure and/or chemotherapy. More particularly, the present invention relates to a therapeutic composition comprising a histone deacetylase inhibitor formulated with a biocompatible polymer that is useful for treating or preventing dermatitis and mucositis in cancer therapy. The present invention also relates to the use histone deacetylase inhibitors to treat and prevent gastrointestinal distress, cancer-related fatigue syndrome and cachexia that are associated with mucositis in cancer therapy

2. Description of the Related Art

A. Chemotherapy and Radiation-induced Epithelial Lining Tissue Damage.

One complication of radiation therapy and chemotherapy is the damage that occurs in the epithelial lining tissues including skin and mucosa. This kind of damage to skin and mucosa is called dermatitis and mucositis, respectively. Severe oral mucositis is especially common among patients who receive aggressive myeloablative chemotherapy for haematopoietic stem-cell transplant, and patients with head and neck cancers receiving radiotherapy and chemotherapy.

It has become clear that instead of epithelial damage simply arising from the direct effects of chemotherapy and/or radiotherapy on basal epithelial stem cells, epithelial lining tissue damage appears to be the consequence of a sequence or series of biological events that begin in the connective tissue (endothelial and mesenchymal cells) of submucosa and target the epithelial cells (Sonis S T et al., J. Supp. Oncol. 2:21-31, 2004). The pathogenesis of epithelial damage induced by radiotherapy and chemotherapy can be thought of as occurring in five phases:

Phase I—Initiation. Initiation of chemotherapy and radiation-induced cell damage is characterized by generation of reactive oxygen species (ROS) to break double-strand DNA, and coincidence of activation of ROS-independent signal pathways such as protein kinase c (PKC).

Phase II—Damage Message Generation. Damage message generation is characterized by activation of transcriptional factors such as NF-κB to turn on pro-inflammatory cytokine expression such as TNF-α, IL-1β, and IL-6.

Phase III—Damage Signal and Amplification. Damage signal amplification by positive feedback loops between NF-κB and TNF-α further increases the numbers and levels of pro-inflammatory cytokines; TNF-α not only further increases the activity of NF-κB but also induces the extrinsic apoptotic pathway resulting in epithelial cell death.

Phase IV—Ulceration and Infection. Ulceration and infection (moderate to severe mucositis) characterized by primary loss of epithelial cells and secondary colonization of bacteria (causing pain, inflammation and loss of function);

Phase V—Healing. The healing process of re-epithelium is stimulated by signals from the exposed extracellular matrix and growth factors secreted from the fibroblasts in the submucosa.

Although there might be some skin or mucosal erythema during phase I to III, tissue integrity is still in place and patients have few symptoms until ulcerative wound develops due to epithelial cell death in phase IV. The process of epithelial lining tissue injury from initiation (phase I) to healing (phase V) is believed to recur at different sites on the skin and mucosa following each fraction of radiotherapy or each cycle of chemotherapy throughout the whole treatment course. Thus, each of the five phases previously described offers potential targets for the prevention, amelioration, and/or acceleration of healing of epithelial damage induced by chemotherapy and/or radiation.

However, there are some concerns that anti-oxidant agents to decrease ROS in phase I and growth factors to promote epithelial growth in phase V might protect normal tissue from radiation and chemotherapy-induced injury while they also enhance tumor growth in cancer therapy. Therefore; it seems that only phase II (such as NF-kappaB) and phase III (such as TNF-alpha) are better targets for treatment and prevention of dermatitis and mucositis without compromising tumor control.

Once chemotherapy and radiation cause epithelial lining tissue damage, the production of cytokines such as TNF-α and growth factors such as TGF-β in the irradiated tissues perpetuates and augments the inflammatory response, while promoting fibroblast recruitment and proliferation but inhibiting epithelial cell growth, resulting in epithelial cell loss such as mucositis and dermatitis, and tissue fibrosis. (Hill, R P., et al., Int. J. Radiat. Oncol. Biol. Phys., 49: 353-365, 2001). The amplified injury response to chemotherapy and radiation by the persistent secretion of TNF-α and TGF-β from epithelial, endothelial, macrophages, and connective tissue cells is possibly caused by a modification in the genetic programming of cell differentiation, proliferation, DNA repair, clonogenic cell death and apoptosis (Zhou, D., et al., Int. J. Radiat. Biol., 77: 763-772, 2001). Thus, the chemotherapy and radiation-induced epithelial lining tissue injury could be regarded as a genetic disorder in the wound healing process.

B. Histone Deacetylase (HDAC) Inhibitors

HDAC inhibitors as a class of compounds with abilities in multiple gene regulation can modulate the expression of a specific set of genes by increasing histone acetylation, thereby regulating chromatin structure and accessibility of target genes for transcription and thus treating diseases (Marks, P A., et al., J. Natl. Cancer Inst., 92: 1210-6, 2000). HDAC inhibitors act selectively on gene expression, altering the expression of only about 2% of the genes expressed in cultured tumor cells. By modulating specific genes related to cell cycle control, DNA repair, tumor suppression, apoptosis and oncogenesis, HDAC inhibitors have shown to be potent inducers of growth arrest, differentiation, and/or apoptotic cell death of transformed cells in vitro and in vivo. Although the effects of HDAC inhibitors induce bulk histone acetylation, they result in apoptotic cell death, terminal differentiation, and growth arrest only in tumor cells but no toxicity in normal cells (Richon, V M., et al., Proc. Natl. Acad. Sci. USA., 97: 10014-10019, 2000; Van Lint, C., et al., Gene Expr., 5: 245-243, 1996). In addition, the modulation of chromatin conformation by HDAC inhibitors can further radiosensitize tumors whose cells are intrinsically radioresistant (Ferrandina, G., et al., Oncol. Res., 12: 429-440, 2001; Miller, A C., et al., Int. J. Radiat. Biol., 72: 211-218, 1997; Biade, S., et al., Int. J. Radiat. Biol., 77: 1033-1042, 2001). The epigenetic modification of chromatin structure for gene regulation suggests that HDAC inhibitors could be therapeutic candidates not only for cancers but also for genetic disorders (Jaenisch, R., et al., Nat. Genet., 33: 245-254, 2003; Garber, K., et al, J. Natl. Cancer Inst., 94: 793-795, 2002). On the other hand, HDAC inhibitors can also induce non-histone protein hyperacetylation. The hyperacetylation of nonhistone proteins such as ribosomal S3 or the Rel-A subunit of NF-κB will inhibit the NF-κB activity and suppress the pro-inflammatory cytokine production (Chen, L., et al., Science, 293: 1653-1657, 2001). In addition to the anti-cancer effects, HDAC inhibitors have also demonstrated the anti-inflammatory effects in many inflammation diseases such as ulcerative colitis and autoimmune diseases (Segain, J P., et al., Gut, 47: 397403, 2000; Mishra, N., et al., Proc. Natl. Acad. Sci. USA., 98: 2628-2633, 2001; Leoni, F., et al., Proc. Natl. Acad. Sci. USA, 99: 2995-3000, 2002; Chung, Y L., et al., Mol. Ther. 8: 707-717, 2003).

On the basis of the potential possibility in simultaneously, coordinately, selectively, and epigenetically manipulating the expression of tumor suppressors, oncogenes, pro-inflammatory cytokines (TNF-α), and fibrogenic growth factors (TGF-β) by differentially remodeling the chromatins in normal and tumor cells, the use of HDAC inhibitors could decrease chemotherapy and radiotherapy-induced epithelial lining tissue toxicities such as mucositis and dermatitis without compromising the tumor killing to maximize the therapeutic effectiveness of cancer therapy

C. Gastrointestinal Distress, Cancer-related Fatigue Syndrome and Cachexia That are Associated With Mucositis Induced by Radiation or Chemotherapy

Gastrointestinal (GI) distress such as nausea, vomiting and diarrhea, cancer-related fatigue syndrome and cachexia that are all associated with mucositis induced by radiotherapy and chemotherapy cause a significant deterioration in the quality of life as well as physical and cognitive functioning, resulting in delay or interruption of potentially curative therapy.

Chemotherapy and/or radiotherapy induce GI distress, in part, by causing enterochromaffin cells lining the GI tract or mucosa in response to cell damage (mucositis) to release serotonin and other neuroactive agents to bind to their receptors in afferent vagal nerves in the GI tract or mucosa and send impulses to the vomiting center (VC) and the chemoreceptor trigger zone (CTZ) in the parvicellular reticular formation in the lateral medullary region of the brain stem and the area postrema near the 4^{th} ventricle of central nervous system (CNS), respectively (Navari R M. J. Supp. Oncol. 1:89-92, 2003; Grunberg S M. J. Supp. Oncol. 2:1-12, 2004). Activation of the CTZ also triggers the release of neurotransmitters that further activate the VC. The CTZ neurotransmitters that are thought to relate to chemotherapy and radiotherapy-induced GI distress include, but are not limited to, dopamine, serotonin, histamine and norepinephrine. Direct links exist between the higher CNS centers and the VC/CTZ. The efferent branches of cranial nerves V, VII and IX, as well as the vagus nerve and sympathetic trunk then produce the complex coordinated set of muscular contractions, cardiovascular responses and reverse peristalsis that characterize vomiting.

However, only to block the actions of neurotransmitters in the GI tract and CNS has been shown to be ineffective to treat all types of chemotherapy and radiotherapy-induced GI distress, and causes significant side effects. Thus, in addition to target the active neurotransmitters and their receptors in the GI tract and the CNS, an approach or agent that can prevent the cell damage (mucositis) to decrease the release of neurotransmitters from the GI tract to the vomiting centers of CNS may be useful in preventing and treating chemotherapy and radiotherapy-induced GI distress.

On the basis of the potential abilities in preventing epithelial lining tissue damage from chemotherapy and radiotherapy, a pharmaceutical composition comprising the HDAC inhibitor could result in preventing the mucositis of GI tract and decreasing the afferent vagal inputs from the GI tract to the VC/CTZ of CNS to prevent or treat chemotherapy and radiotherapy-induced GI distress.

Cancer-related fatigue, a subjective state of overwhelming and sustained exhaustion and decreased capacity for physical and mental work that is not relieved by rest, is generally protracted, cumulative, and progressive. A complex network of proinflammatory cytokines triggered by the malignancy and mucositis induced by radiotherapy and chemotherapy is thought to be involved in the development of cancer-related fatigue, as well as in the accompanying cachexia, which is characterized by loss of adipose tissue and skeletal muscle mass (Argiles J M, et al. Med Res Rev 12:637-652, 1992). Central to this network is TNF-alpha, a cytotoxic cytokine secreted by macrophages in response to tumor invasion, and mucositis induced by radiotherapy and chemotherapy.

Therefore, by reducing TNF-alpha expression and mucositis in cancer therapy, the HDAC inhibitors may decrease the GI distress, cancer-related fatigue syndrome and cachexia associated with mucositis.

### BRIEF SUMMARY OF INVENTION

A detailed description is given in the following embodiments with reference to the accompanying drawings.

In accordance with the present invention, there is provided a composition and method for treating or preventing epithelial lining tissue damage from dermatitis or mucositis induced by radiation exposure and/or chemotherapy in a subject receiving cancer therapy. The invention also provides a method for treatment or prevention of GI distress, cancer-related fatigue syndrome and cachexia that are associated with mucositis in cancer therapy. By treatment of dearmatitis or mucositis, it is meant that the therapeutic composition is effective to prevent or reduce the incidence, severity and/or duration of the disease.

The therapeutic composition comprises an HDAC inhibitor that, as formulated in the therapeutic composition, presents therapeutic effect in mammalian hosts, typically human hosts receiving cancer therapy, for the treatment of dermatitis or mucositis, together with at least one biocompatible polymer or carrier that aids delivery, prolongs retention, and sustains action of the HDAC inhibitor to the targeted skin, mucosa or other tissues of the body and adheres to skin, mucosa or other tissues of the body to promote effective delivery of the HDAC inhibitor to treat the targeted skin and mucosal sites. One preferred embodiment of the therapeutic composition includes phenylbutyrate as the pharmaceutical substance and at least one biocompatible gel-forming polymer.

For many applications, gel-forming polymers are beneficial to produce coating on the mucosal surface and which will allow longer contact of active to the mucosa where also the coating will mechanically protect mucosa from possible irritation from possible irritation.

Not all aspects of the invention when treating for dermatitis or mucositis are so limited to topical gel forms. Depending on the area of delivery the HDAC inhibitor of the present invention can be formulated in different product forms. The present invention also involves use of the therapeutic composition that can be administered intraperitoneally, intrathecally, intraarterially, intranasally, intraparenchymally, subcutaneously, intramuscularly, intravenously, dermally, and intrarectally. The dosage amounts are based on the effective concentration observed in vitro and in vivo studies.

In another aspect, the invention involves a therapeutic composition useful for treatment of GI distress, cancer-related fatigue syndrome and cachexia that are associated with mucositis induced by radiotherapy and chemotherapy, with the composition administered concomitantly or in combination with a 5-hydroxytryptamine₃ (5-HT₃) receptor antagonist, a dopamine receptor antagonist, a DOPA-5-HT₃ receptor antagonist, a neurokinin (NK)-1 receptor antagonist, an anti-histamine, an anticholinergics, a non-steroid anti-inflammation drug, a steroid, a growth factor, an anti-oxidant agent, a tricyclic antidepressant, a sedative agent, cannabinoids, an inflammatory cytokine inhibitor, a mast cell inhibitor, an MMP inhibitor, an NO inhibitor, an antibiotics, a vitamin, a histone deacetylase inhibitor, an anesthetics, scuralfate, or an inhibitor of NF-kappaB.

Both the foregoing general description and the following detailed description are exemplary and explanatory and are intended to provide explanation of the invention as claimed. Other objects, advantages and novel features will be readily apparent to those skilled in the art from the following detailed description of the invention.

### BRIEF DESCRIPTION OF DRAWINGS

The present invention can be more fully understood by reading the subsequent detailed description and examples with references made to the accompanying drawings, wherein:

FIG. 1 is a diagram of the clinical mucositis scores in the hamster buccal pouch following acute radiation and application of ASN-02 oral gel (phenylbutyrate, an HDAC inhibitor formulated with a biocompatible polymer to form a reverse-thermal gelation polymer gel). The vehicle (only polymer) and blank control groups exhibit the expected clinical severe radiation-induced mucositis scores at the expected peak mucositis time at Day 14 to Day 18 following radiation, and the ASN-02 oral gel reduces the mean clinical mucositis scores relative to the vehicle and blank controls in incidence, severity and duration of radiation-induced mucositis; and

FIGS. 2A to 2F are gross pictures showing that at Day 14 following radiation, the huge difference between the blank and vehicle control groups and the ASN-02-treated group. Ulcerative mucositis appears at the blank and vehicle groups; in contrast, the buccal mucosa is still intact in the ASN-02-treated group. FIGS. 2A and 2B are the ASN-02-treated group; FIGS. 2C and 2D the blank control group; FIGS. 2E and 2F the vehicle control group. FIGS. 2A, 2C, and 2E are at Day 6; FIGS. 2B, 2D, and 2F at Day 14.

FIG. 3 is a diagram showing that temporal variation in mRNA levels of TNF-α in mucosa after irradiation, normalized to the internal control GAPDH and expressed as a ratio to levels in nonirradiated mucosa samples. Each point represents the mean of mRNA levels of 5 samples in the same group of blank, vehicle, or ASN-02 oral gel. The timing of peak appearance of TNF-α upregulation correlated well with the development of mucositis. ASN-02 oral gel effectively suppressed the long-term aberrant expression of TNF-α when compared to the blank and vehicle control.

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

A class compound of gene modulators, HDAC inhibitors, activates and represses a subset of genes by remodeling the chromatin structure via the altered status in histone acetylation (Marks et al, J. Natl. Cancer Inst., 92: 1210-6, 2000; Kramer et al, Trends Endocrinol. Metab., 12: 294-300, 2001). Histone hyperacetylation results in the up-regulation of cell-cycle inhibitors (p21^{Cip1}, p27^{Kip1}, and p16^{INK4}), the down-regulation of oncogenes (Myc and Bcl-2), the repression of inflammatory cytokines (interleukin (IL-1, IL-8, TNF-α, and TGF-β), or no change (GAPDH and γ-actin)(Lagger et al, EMBO J., 21: 2672-81, 2002; Richon et al, Clin. Cancer Res., 8: 662-667, 2002; Richon et al, Proc. Natl. Acad. Sci. USA., 97: 10014-9, 2000; Van Lint et al, Gene Expr., 5: 245-3, 1996; Huang et al, Cytokine, 9: 27-36, 1997; Mishra et al, Proc. Natl. Acad. Sci. USA., 98: 2628-33, 2001; Stockhammer et al, J. Neurosurg., 83: 672-81, 1995; Segain et al, Gut, 47: 397-403, 2000; Leoni et al, Proc. Natl. Acad. Sci. USA, 99: 2995-3000, 2002). In addition to inducing histone hyperacetylation, HDAC inhibitors also induce hyperacetylation of nonhistone proteins such as ribosomal S3, p53 or the Rel-A subunit of NF-κB, modulate protein kinase C (PKC) activity, inhibit protein isoprenylation, decrease DNA methylation, and bind to nuclear receptors (Webb et al, J. Biol. Chem., 274: 14280-7, 1999; Chen et al, Science, 293: 1653-7, 2001). More and more different mechanisms also pointed to inhibition of NF-κB transcriptional activity after treatment with HDAC inhibitors. HDAC inhibitors have exhibited properties in inducing cell-cycle arrest, cell differentiation, and apoptotic cell death in tumor cells and in decreasing inflammation and fibrosis in inflammatory diseases (Warrell et al, J. Natl. Cancer Inst., 90: 1621-5, 1998; Vigushin et al, Clin. Cancer Res., 7: 971-6, 2001; Saunders et al, Cancer Res., 59: 399-404, 1999; Gottlicher et al, EMBO J., 20: 6969-78, 2001; Rombouts et al, Acta Gastroenterol. Belg., 64: 239-46, 2001). Although the effects of HDAC inhibitors induce bulk histone acetylation, they result in apoptotic cell death, terminal differentiation, and growth arrest in tumor cells but not in normal cells because the sensitivity to apoptosis induction by HDAC inhibitors depends on the original state of cell differentiation as well as the acetylated histone status (Garber et al, J. Natl. Cancer Inst., 94: 793-5, 2002). In addition, the modulation of chromatin conformation by HDAC inhibitors can further radiosensitize tumors whose cells are intrinsically radioresistant, and also sensitize tumor cells to chemotherapy (Ferrandina et al, Oncol. Res., 12: 429-40, 2001; Miller et al, Int. J. Radiat. Biol., 72: 211-8, 1997; Biade et al, Int. J. Radiat. Biol., 77: 1033-42, 2001).

On the basis of the potential possibility in simultaneously, coordinately, selectively, and epigenetically manipulating the expression of tumor suppressors, oncogenes, and pro-inflammatory cytokines (TNF-α, IL-1, IL-6) by differentially remodeling the chromatins and inhibiting the NF-κB activity in normal and tumor cells, it is likely that HDAC inhibitors can exert their effects on the attenuation of epithelial lining tissue damage from dermatitis and mucositis by interfering with the phase II/III of pathogenesis of chemotherapy and radiotherapy-induced epithelial damage (Sonis S T et al., J. Supp. Oncol. 2:21-31, 2004), and still exhibit anti-tumor effects and tumor radiosensitization in cancer treatment.

Active pharmaceutical compounds used to carry out the present invention are, in general, histone hyperacetylating agents, such as HDAC inhibitors. Numerous such pharmaceutical compounds are known. See, e.g., P. Dulski, Histone Deacetylase as Target for Antiprotozoal Agents, PCT Application WO 97/11366 (Mar. 27, 1997). Examples of such compounds include, but are not limited to:

A. Trichostatin A and its analogues such as: Trichostatin A (TSA); and Trichostatin C (Koghe et al. 1998. Biochem. Pharmacol. 56:1359-1364) (Trichostatin B has been isolated but not shown to be an HDAC inhibitor).

B. Peptides, such as: Oxamflatin [(2E)-5-[3-[(phenylsufonyl)aminol phenyl1]-pent-2-en-4-ynohydroxamic acid (Kim et al. Oncogene, 18:2461-2470 (1999)); Trapoxin A (TPX)--Cyclic Tetrapeptide (cyclo-(L-phenylalanyl-L-phenylalanyl-D-pipecolinyl-L-2-amino-8-oxo-9,10--epoxy-decanoyl)) (Kijima et al., J. Biol. Chem. 268, 22429-22435 (1993)); FR901228, Depsipeptide (Nakajima et al., Ex. Cell Res. 241, 126-133 (1998)); FR225497, Cyclic Tetrapeptide (H. Mori et al., PCT Application WO 00/08048 (Feb. 17, 2000)); Apicidin, Cyclic Tetrapeptide [cyclo(N--O-methyl-L-tryptophanyl-L-isoleucinyl-D-pipecolinyl-L-2-amino-8--oxodecanoyl)] (Darkin-Rattray et al., Proc. Natl. Acad. Sci. USA 93, 13143-13147 (1996)); Apicidin 1a, Apicidin Ib, Apicidin Ic, Apicidin IIa, and Apicidin IIb (P. Dulski et al., PCT Application WO 97/11366); HC-Toxin, Cyclic Tetrapeptide (Bosch et al., Plant Cell 7, 1941-1950 (1995)); WF27082, Cyclic Tetrapeptide (PCT Application WO 98/48825); and chlamydocin (Bosch et al., supra).

C. Hydroxamic Acid-Based Hybrid Polar Compounds (HPCs), such as: Salicylihydroxamic Acid (SBHA) (Andrews et al., International J. Parasitology 30, 761-8 (2000)); Suberoylanilide Hydroxamic Acid (SAHA) (Richon et al., Proc. Natl. Acad. Sci. USA 95, 3003-7 (1998)); Azelaic Bishydroxamic Acid (ABHA) (Andrews et al., supra); Azelaic-1-Hydroxamate-9-Anilide (AAHA) (Qiu et al., Mol. Biol Cell 11, 2069-83 (2000)); M-Carboxycinnamic Acid Bishydroxamide (CBHA) (Ricon et al., supra); 6-(3-Chlorophenylureido)carpoic Hydroxamic Acid (3-Cl-UCHA) (Richon et al., supra); MW2796 (Andrews et al., supra); and MW2996 (Andrews et al., supra). Note that analogs not effective as HDAC Inhibitors are: Hexamethylene bisacetamide (HBMA) (Richon et al. 1998, PNAS, 95:3003-7); and Diethyl bix(pentamethylene-N,N-dimethylcarboxami-de) malonate (EMBA) (Richon et al. 1998, PNAS, 95:3003-7).

D. Short Chain Fatty Acid (SCFA) Compounds, such as: Isovalerate (McBain et al., Biochem. Pharm. 53:1357-68 (1997)); Valproic acid; Valerate (McBain et al., supra); 4-Phenylbutyrate (4-PBA) (Lea and Tulsyan, Anticancer Research, 15, 879-3 (1995)); Phenylbutyrate (PB) (Wang et al., Cancer Research, 59, 2766-99 (1999)); Propionate (McBain et al., supra); Butrymide (Lea and Tulsyan, supra); Isobutyramide (Lea and Tulsyan, supra); Phenylacetate (Lea and Tulsyan, supra); 3-Bromopropionate (Lea and Tulsyan, supra); Arginine butyrate (Shao et al., Leukemia Research, 26, 1077-83 (2002); and Tributyrin (Guan et al., Cancer Research, 60, 749-55 (2000)).

E. Benzamide derivatives, such as: MS-27-275 [N-(2-aminophenyl)-4-[N-(pyridin-3-yl-methoxycarbonyl) aminomethyl]benzamide] (Saito et al., Proc. Natl. Acad. Sci. USA 96, 4592-7 (1999)); and 3′-amino derivative of MS-27-275 (Saito et al., supra).

F. Other inhibitors, such as: Depudecin [its analogues (mono-MTM-depudecin and depudecin-bisether) do not inhibit HDAC] (Kwon et al. 1998. PNAS 95:3356-61); and Scriptaid (Su et al. 2000 Cancer Research, 60:3137-42).

Histone deacetylases (HDACs) as that term is used herein are enzymes which catalyze the removal of acetyl groups from lysine residues in the amino terminal tails of the nucleosomal core histones. As such, HDACs together with histone acetyl transferases (HATs) regulate the acetylation status of histones. Histone acetylation affects gene expression and inhibitors of HDACs, such as the hydroxamic acid-based hybrid polar compound suberoylanilide hydroxamic acid (SAHA) induce growth arrest, differentiation and/or apoptosis of transformed cells in vitro and inhibit tumor growth in vivo. HDACs can be divided into three classes based on structural homology. Class I HDACs (HDACs 1, 2, 3 and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein, and have both nuclear and cytoplasmic subcellular localization. Both Class I and II HDACs are inhibited by hydroxamic acid-based HDAC inhibitors, such as SAHA. Class III HDACs form a structurally distant class of nicotinamide (NAD) dependent enzymes that are related to the yeast SIR2 proteins and are not inhibited by hydroxamic acid-based HDAC inhibitors.

HDAC inhibitors, as that term is used herein are compounds which are capable of inhibiting the deacetylation of histones in vivo, in vitro or both. As such, HDAC inhibitors inhibit the activity of at least one histone deacetylase. As a result of inhibiting the deacetylation of at least one histone, an increase in acetylated histone occurs and accumulation of acetylated histone is a suitable biological marker for assessing the activity of HDAC inhibitors. Therefore, procedures which can assay for the accumulation of acetylated histones can be used to determine the HDAC inhibitory activity of compounds of interest. It is understood that compounds which can inhibit histone deacetylase activity can also bind to other substrates and as such can inhibit other biologically active molecules such as enzymes or non-histone proteins.

The HDAC inhibitor agents can be brought in the form of pharmaceutically acceptable salts. As such pharmaceutically acceptable salts may be used so long as they do not adversely affect the desired pharmacological effects of the compounds. The selection and production can be performed by those skilled in the art. Examples of pharmaceutically acceptable salts include alkali metal salts such as sodium salt or a potassium salt, alkaline earth metal salts such as calcium salt or a magnesium salt, salts with an organic base such as an ammonium salt, or a salt with an organic base such as a triethylamine salt or an ethanolamine salt.

The therapeutic composition of the present invention may be administered orally or non-orally. In the case of oral administration, they may be administered in the form of soft and hard capsules, tablets, granules, powders, solutions, suspensions, mouth wash or the like. In the case of non-oral administration, they may be administered in the form of creams, ointments, gels, lotions, patches, suppositories, liposome formations, injection solution, eye drops, nasal spray, pulmonary inhalants, drip infusion formulations, enema or the like whereby continued membrane absorption can be maintained in the form of solid, viscous liquid, bioadhesive substance or suspension. The selection of the method for the preparation of these formulations and the vehicles or carriers, disintegrators or suspending agents, can be readily made by those skilled in the art.

More particularly, the therapeutic compositions of the present invention may contain HDAC inhibitors with pharmaceutically acceptable biocompatible polymers thereof. In one aspect, the present invention provides a therapeutic composition that comprises an HDAC inhibitor formulated with at least one, and optionally more than one, a biocompatible polymer for delivery of therapeutics to humans with cancers receiving radiotherapy and/or chemotherapy, especially for use when bioadhesion or prolonged and sustained action of the therapeutic(s) is desired, thereby improving the efficacy of the dermatitis and mucositis therapeutic upon topical application to skin and mucosal surfaces, a route that may otherwise be an ineffective means of therapy. Furthermore, the delivery system may reduce the frequency and duration of administration of the dermatitis and mucositis therapeutic as part of a treatment.

Each therapeutic for GI distress, cancer-related fatigue syndrome and cachexia that are associated with mucositis in cancer therapy is an HDAC inhibitor formulated with or without at least one biocompatible polymer or carrier that provides a therapeutic effect, either alone or in combination with other agents, including a 5-hydroxytryptamine₃ (5-HT₃) receptor antagonist, a dopamine receptor antagonist, a DOPA-5-HT₃ receptor antagonist, a neurokinin (NK)-1 receptor antagonist, an anti-histamine, an anticholinergics, a non-steroid anti-inflammation drug, a steroid, a growth factor, an anti-oxidant agent, a tricyclic antidepressant, a sedative agent, cannabinoids, an inflammatory cytokine inhibitor, a mast cell inhibitor, an MMP inhibitor, an NO inhibitor, an antibiotics, a vitamin, a histone deacetylase inhibitor, an anesthetics, sucralfate, and an inhibitor of NF-kappaB. In that regard, the therapeutic effect may be due to the direct action of the HDAC inhibitor of the composition, or may be due to one or more other agents having synergistical effects with the HDAC inhibitor. The amount of HDAC inhibitor in each therapeutic in combination with other agents varies depending on the nature and potency of the therapeutic.

Effective amounts of the HDAC inhibitors and different treatment regimens of HDAC inhibitors combined with other agents for any particular subject (e.g., human, dog, or cat) will also depend upon a variety of other factors, including the activity of the specific compound employed, age, body weight, general health status, sex, diet, time of administration, rate of excretion, severity and course of the disease, and the patient's disposition to the disease, but the amount of HDAC inhibitor is usually from 0.001% to 100% by weight of the composition irrespective of the manner of administration. The other agents may optionally be administered concurrently or sequentially. As used herein, the word “concurrently” means sufficiently close in time to produce a combined effect (that is, concurrently may be simultaneously, or it may be two or more events occurring within a short time period before or after each other). As used herein, the administration of two or more compounds “concurrently” or “in combination” means that the two compounds are administered closely enough in time that the presence of one alters the biological effects of the other. The two compounds may be administered simultaneously or sequentially. Simultaneous administration may be carried out by mixing the compounds prior to administration, or by administering the compounds at the same point in time but at different anatomic sites or using different routes of administration.

The therapeutic compositions in the present invention can be in any convenient physical form, but are often preferably in the form of a flowable fluid medium at the time of administration. For example, when treating for alimentary mucositis, the therapeutic composition is preferably sufficiently fluid in character that it can be accepted in the oral cavity and swished in the manner of a mouthwash, or swallowed. In this situation, the therapeutic composition will typically include as its largest constituent a carrier liquid to impart the flowable fluid properties to the therapeutic composition. In most instances the carrier liquid will be water. The HDAC inhibitor and the biocompatible polymer are each dissolved in the carrier liquid or suspended in the carrier liquid as a disperse phase. For example, the therapeutic composition can comprise an aqueous solution of the biocompatible polymer, with the HDAC inhibitor also dissolved in the solution and/or suspended as a precipitate in the solution. Preferably, both of the biocompatible polymer and the HDAC inhibitor are dissolved in the carrier liquid, at least at a temperature at which the therapeutic composition is to be administered to patients. Having the biocompatible polymer and the HDAC inhibitor codissolved in the carrier liquid ensures intimate mixing of the two materials, which promotes adhesion of the HDAC inhibitor to surfaces of the skin or mucosa along with the biocompatible polymer, thereby effectively using the therapeutic. Proper selection of the biocompatible polymer is important to enhanced performance of therapeutic composition. The concentration of the biocompatible polymer in the composition will vary depending upon the specific biocompatible polymer and the specific situation. In most situations, however, the biocompatible polymer will comprise from about 1% by weight to about 70% by weight, and more typically from about 5% by weight to about 20% by weight of the therapeutic composition.

In one important embodiment, the biocompatible polymer is selected so that when the biocompatible polymer is incorporated into the therapeutic composition, the viscosity of the therapeutic composition increases with increasing temperature in the vicinity of physiological temperature, which is typically about 37.degree. C. In this way, the therapeutic composition can be administered as a lower viscosity flowable fluid medium at a cool temperature, and the viscosity of the therapeutic composition will increase as the therapeutic composition is warmed to physiological temperature. In one preferred embodiment for many applications when it is desirable for the therapeutic composition to exhibit reverse-thermal viscosity behavior, the therapeutic composition exhibits reverse-thermal viscosity behavior over at least some range of temperatures between 1.degree. C. and the physiological temperature of the host (e.g., 37.degree. C. for a human host). Biocompatible polymers that may be used to make the therapeutic composition of the present invention include, in general, a bioadhesive polymer, a cationic polymer, a viscous polymer gel, a hydrogel, a natural polymer, a polyoxyalkylene block copolymer, and a reverse-thermal gelation polymer. The therapeutic composition may include, in addition to the biocompatible polymer, a separate bioadhesive agent that enhances the bioadhesive properties of the therapeutic composition. The bioadhesive agent is frequently a second polymer having even greater bioadhesive properties.

The therapeutic composition can be further formulated with sucralfate in a thicker gel form, that can be spooned into the mouth and swallowed, and coat the whole GI tract. Depending on the area of delivery the pharmaceutical substance of the present invention can also be formulated in different product forms. Some examples of possible product forms for administration of the therapeutic composition include an oral solution, bladder irrigation solution, gel, slurry, mouthwash, lozenge, tablet, film, patch, lollipop, spray, drops or suppository. For example, a gel formulated into a suppository would be one preferred product form for administration to treat mucosal surfaces of either the rectum or the vagina. A tablet, patch or film could be formulated to administer the therapeutic composition orally or sublingually. A slurry or oral solution could be used for treatment of mucosal surfaces in the oral cavity, esophagus and/or GI tract. A bladder irrigation solution would be administered to the bladder by catheter. A spray would be advantageous in delivering the present invention to either the nasal cavity or the lungs, while a droplet formulation would be advantageous for delivery to the eye or inner ear.

In order that the invention described herein may be more readily understood, the following examples are set forth. It should be understood that the examples are for illustrative purpose only and are not to be construed as limiting this invention in any manner. All references cited herein are expressly incorporated by reference in their entirety.

### EXAMPLE 1

### Therapeutic Composition Containing an HDAC Inhibitor Formulated with a Biocompatible Polymer

An approach to selectively reduce mucosa morbidity without compromising the tumor-killing effects of chemotherapy and radiotherapy is a long-sought goal in cancer treatment. This example is to reveal a biologically based, topically applied regimen for treating mucositis. The vehicle acceptability and contact time of the medication in the mucosa are critical to the outcome of pharmacologic agents for treating mucositis. However, there are very few available vehicles or carriers for such an approach.

An HDAC inhibitor, phenylbutyrate, is formulated with a biocompatible reverse-thermal gelation polymer to form a 5% phenylbutyrate oral gel. This feature allows the oral gel to undergo a phase transition, from a liquid upon oral intake, to a gel upon reaching body temperature in the body. This phase transition serves to increase contact time of the active compound phenylbutyrate with the mucosa, through the deposition of a thin coating on all contacted surfaces. An oral gel formulation can be selected because of its ease of use, broad coating of the mucosa, and patient acceptability/familiarity.

For example, each ml of the oral gel contains 50 mg sodium phenylbutyrate, 1.0% gel-forming polymer as bioadhesive substance, methylparaben as preservative, sodium saccharin as sweetener, a fragrant agent and purified water. This product is a transparent, colorless, and gel-like viscous solution.

### EXAMPLE 2

### Treatment of Radiation-Induced Mucositis Using an HDAC Inibitor Formulated With a Biocompatible Polymer

To evaluate the efficacy of the oral gel (ASN-02) containing 5% phenylbutyrate formulated with a biocompatible gel-forming polymer for the treatment of radiation-induced mucositis, a hamster animal model developed by Dr. Steve Sonis (Harvard School of Dental Medicine, Brigham and Women's Hospital, Boston, Mass.) was used.

Male Golden Syrian hamsters, 5 to 6 weeks of age, weighing approximately 90 g at study commencement were anesthetized with an intraperitoneal injection of sodium pentobarbital (80 mg/kg). The left buccal pouch was everted, fixed and isolated using a lead shield. Mucositis was induced using a standardized acute radiation protocol. A single dose of radiation (40 Gy/dose) was administered to the left buccal pouch mucosa of all animals on Day 0. Radiation was generated with a linear accelerator delivering a 6 MeV electron beam at a SSD of 100 cm at a rate of 300 cGy/minute. This radiation protocol produces ‘peak’ oral mucositis at Day 14 to 18 after irradiation. Animals were dosed topically 3 times per day by applying 50 μl of ASN-02 or vehicle into the left (irradiated) buccal pouch per application from Day 1 to Day 28. Clinical mucositis was assessed every day starting on Day 6 to Day 28. Mucositis was evaluated by a visual scoring system. Following visual scoring, a photograph of each animal's mucosa was taken for comparison.

Description of clinical mucositis scoring: A score of 1-2 is considered to represent a mild stage of the mucositis, whereas a score of 3-5 is considered to indicate moderate to severe mucositis.

| Score | Description |
|---|---|
| 0 | Pouch completely healthy. No erythema or vasodilation. |
| 1 | Light to severe erythema and vasodilation. No erosion of mucosa. |
| 2 | Severe erythema and vasodilation. Erosion of superficial aspects |
| | of mucosa leaving denuded areas. Decreased stippling of mucosa. |
| 3 | Formation of off-white ulcers in one or more places. Ulcers may |
| | have a yellow/gray due to pseudomembrane. Cumulative size of |
| | ulcers should equal about ¼ of the pouch. Severe erythema and |
| | vasodilation. |
| 4 | Cumulative size of ulcers should equal about ½ of the pouch. |
| | Loss of pliability. Severe erythema and vasodilation. |
| 5 | Virtually all of pouch is ulcerated. Loss of pliability (pouch |
| | can only partially be extracted from mouth. |

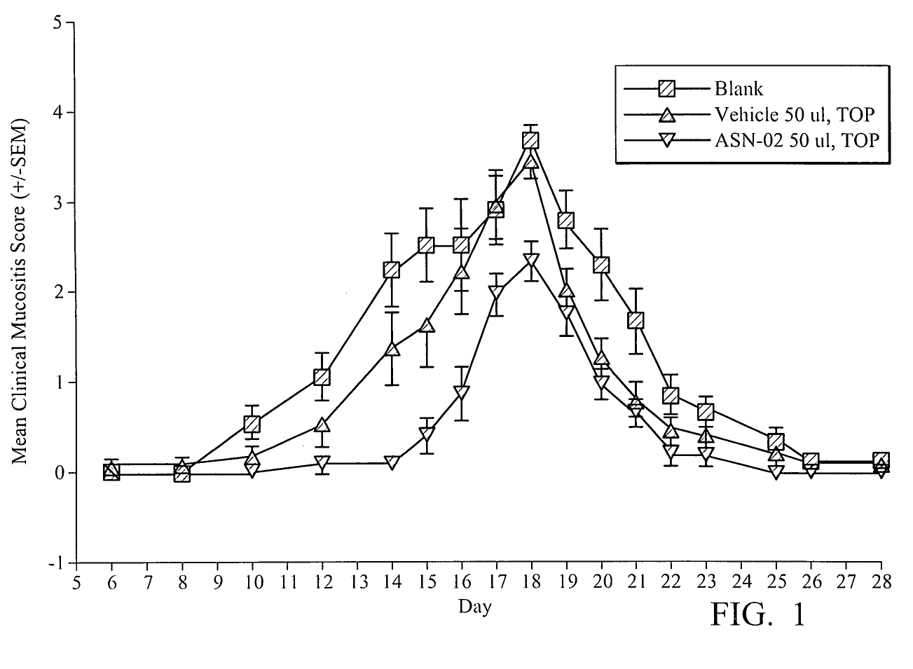

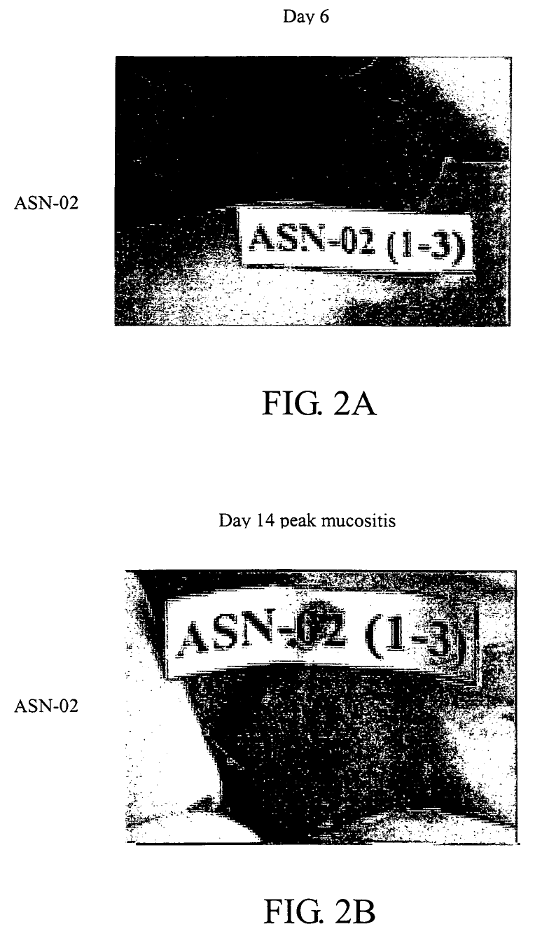

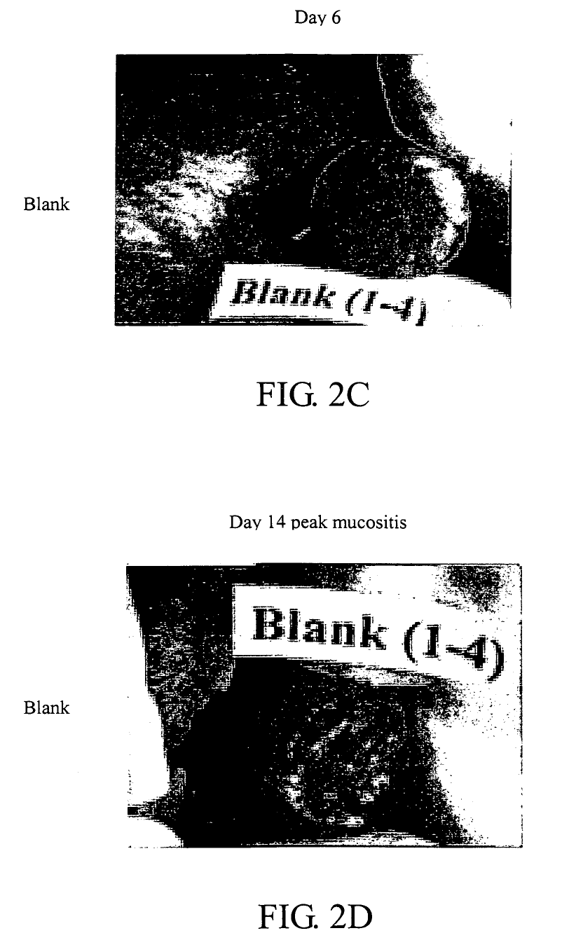

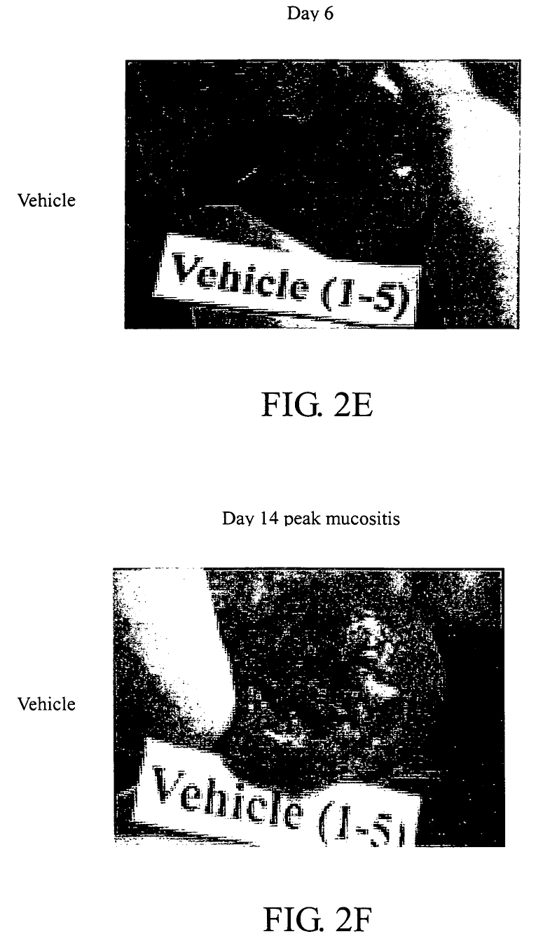

Data of visual scoring of mucositis and representative photographs showing the development of peak mucositis are shown in FIG. 1 and FIG. 2, respectively. Severity of mucositis increases with score. A score of 1-2 is considered to represent a mild stage of the mucositis, whereas a score of 3-5 is considered to indicate moderate to severe mucositis. Values are the mean clinical mucositis scores ±SEM per formulation treatment or control group (N=9 hamsters in the blank control group, N=9 hamsters in the vehicle group, and N=11 hamsters in the ASN-02 group). The vehicle and blank control groups exhibited the expected clinical mucositis score (i.e., a score of 3 to 4) at the expected peak mucositis time (i.e., 14 to 18 days post-irradiation). Five animals of 11 in the ASN-02 group had mucositis scores between 2.0 and 3.0 at the peak (Day 18), and none in the ASN-02 group had score>3.0 during the whole course. All animals (n=9) in the blank control group had mucositis score>3.0 at peak (Day 18), whereas 6 of them had score>=4.0. All animals (n=9) in the vehicle group had mucositis score>3.0 at peak (Day 18), whereas 5 of them had score>=4.0. The mean peak mucositis score was 2.3 in the ASN-02 group compared with 3.75 in the vehicle and blank control groups. In addition to preventing moderate and severe mucositis, ASN-02 improved mild mucositis at the peak to normal appearance in 2 days. Taken together, the ASN-02 (phenylbutyrate) oral gel reduced the mean clinical mucositis scores relative to the vehicle and blank controls in incidence, severity and duration of radiation-induced oral mucositis.

### EXAMPLE 3

### Long-term Suppression of the Radiation-induced Aberrant Proinflammatory Cytokine by an HDAC Inhibitor Formulated With a Biocompatible Polymer

Development of GI distress, cancer-related fatigue syndrome and cachexia associated with mucositis in cancer therapy has been attributed to the radiation and/or chemotherapy-induced persistent up-regulation of proinflammatory cytokines such as TNF-α. Levels of mRNA of the major proinflammatory cytokine, TNF-α, were assessed using a multiple cytokine RNase protection assay kit (Riboquant; Pharmingen, San Diego, Calif.) that contained a template set to allow for the generation of a 32P-labeled antisense RNA probe set that hybridized with the target TNF-α mRNA and the internal control GAPDH. After hybridization of labeled probe to target RNA, unprotected RNA was digested by a ribonuclease (RNase), and protected RNA fragments were resolved on a 6% polyacrylamide gel and recorded by phosphorimaging (Molecular Dynamics Corp., Sunnyvale, Calif.). Densitometry was used to quantify the amount of each mRNA species and was normalized to the internal control GAPDH. The irradiated mucosa (left buccal) and the nonirradiated mucosa (right buccal) were removed for assays at the same time as indicated.

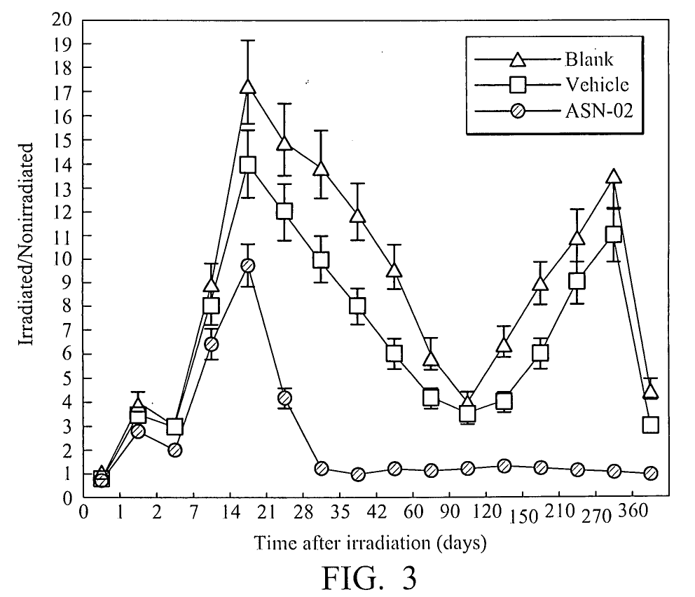

The timing of the peak appearance of TNF-α upregulation induced by radiation correlated with the development of mucositis in all groups as shown in FIG. 3. In the ASN-02-treated group, the highest surge of TNF-α appeared at 1 day after irradiation, but levels were subsequently suppressed after Day 14. The suppression still persisted at 12 months. In the blank and vehicle control groups, mRNA levels of TNF-α in the irradiated mucosa increased and fluctuated above the nonirradiated mucosal levels over a period of 1 year and reached the first peak of 2-3-fold above the nonirradiated mucosal levels at 1 day after irradiation, the second peak of 10.5-16-fold around 14-28 days after irradiation, and the third peak of 13-14-fold at 9 months after irradiation; levels then declined to 2-3-fold normal levels by 12 months after irradiation.

Although each group had body weight loss gradually and there was no significant difference in body weight among the three groups in first 2 weeks, the animals in the ASN-02-treated group regained appetite, activity and body weight significantly sooner than those of the blank and vehicle control groups.

Taken together, these results indicate that the HDAC inhibitor can suppress the long-term upregulation of TNF-A and decrease the radiation-induced acute and chronic side effects, such as mucositis, GI distress, cancer-related fatigue syndrome and cachexia induced.

### OTHER EMBODIMENTS

From the above description, one skilled in the art can easily ascertain the essential characteristics of the present invention, and without departing from the spirit and scope thereof, can make various changes and modifications of the invention to adapt it to various usages and conditions. For example, compounds structurally and functionally analogous to HDAC inhibitors described above can also be used to practice the present invention. Thus, other embodiments are also within the claims.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for treating or preventing epithelial lining tissue damage from mucositis induced by radiation exposure and/or chemotherapy, said method comprising applying to mucosa or other tissues of the body an amount of a therapeutic composition which comprises at least one histone deacetylase (HDAC) inhibitor formulated with or without at least one pharmaceutically acceptable biocompatible polymer or carrier, or pharmaceutically acceptable salts in an amount sufficient to prolong retention and sustain action of the histone deacetylase inhibitor in the mucosa or other tissues of the body to delay onset or decrease severity of the signs and symptoms of mucositis in cancer therapy.

2. The method as claimed in claim 1, wherein the HDAC inhibitor is a hydroxamic acid derivative, a fatty acid derivative, a cyclic tetrapeptide, a benzamide derivative, or an electrophilic ketone derivative.

3. The method as claimed in claim 1, wherein the HDAC inhibitor is valproic acid, trichostatin A, phenylbutyrate, arginine butyrate, depudecin, trapoxin A, depsipeptide, oxamflatin, suberoylanilide hydroxamic acid (SAHA), scriptaid, or MS-27-275.

4. The method as claimed in claim 1, wherein the pharmaceutically acceptable biocompatible polymer or carrier is selected from the group consisting of a bioadhesive polymer, a cationic polymer, a viscous polymer gel, a hydrogel, a natural polymer, a polyoxyalkylene block copolymer, a reverse-thermal gelation polymer, and a liposome.

5. The method as claimed in claim 1, wherein the biocompatible polymer, as formulated in the therapeutic composition, has a gel-forming property.

6. The method as claimed in claim 1, further comprising administering to the subject a second agent selected from a group consisting of a 5-hydroxytryptamine₃ (5-HT₃) receptor antagonist, a dopamine receptor antagonist, a DOPA-5-HT₃ receptor antagonist, a neurokinin (NK)-1 receptor antagonist, an anti-histamine, an anticholinergics, a non-steroid anti-inflammation drug, a steroid, a growth factor, an anti-oxidant agent, a tricyclic antidepressant, a sedative agent, cannabinoids, an inflammatory cytokine inhibitor, a mast cell inhibitor, an MMP inhibitor, an NO inhibitor, an antibiotics, a vitamin, a histone deacetylase inhibitor, an anesthetics, sucralfate or an inhibitor of NF-kappaB.

7. A method for treating or preventing gastrointestinal distress, cancer-related fatigue syndrome and cachexia that are associated with mucositis induced by radiation exposure and/or chemotherapy in cancer therapy, said method comprising applying to mucosa or other tissues of the body an amount of a therapeutic composition which comprises at least one histone deacetylase (HDAC) inhibitor formulated with or without at least one pharmaceutically acceptable biocompatible polymer or carrier, or pharmaceutically acceptable salts in an amount sufficient to prolong retention and sustain action of the histone deacetylase inhibitor in the mucosa or other tissues of the body.

8. The method as claimed in claim 7, wherein the HDAC inhibitor is a hydroxamic acid derivative, a fatty acid derivative, a cyclic tetrapeptide, a benzamide derivative, or an electrophilic ketone derivative.

9. The method as claimed in claim 7, wherein the HDAC inhibitor is valproic acid, trichostatin A, phenylbutyrate, arginine butyrate, depudecin, trapoxin A, depsipeptide, oxamflatin, suberoylanilide hydroxamic acid (SAHA), scriptaid, or MS-27-275.

10. The method as claimed in claim 7, wherein the pharmaceutically acceptable biocompatible polymer or carrier is selected from the group consisting of a bioadhesive polymer, a cationic polymer, a viscous polymer gel, a hydrogel, a natural polymer, a polyoxyalkylene block copolymer, a reverse-thermal gelation polymer, and a liposome.

11. The method as claimed in claim 7, wherein the biocompatible polymer, as formulated in the therapeutic composition, has a gel-forming property.

12. The method as claimed in claim 7, further comprising administering to the subject a second agent selected from a group consisting of a 5-hydroxytryptamine₃ (5-HT₃) receptor antagonist, a dopamine receptor antagonist, a DOPA-5-HT₃ receptor antagonist, a neurokinin (NK)-1 receptor antagonist, an anti-histamine, an anticholinergics, a non-steroid anti-inflammation drug, a steroid, a growth factor, an anti-oxidant agent, a tricyclic antidepressant, a sedative agent, cannabinoids, an inflammatory cytokine inhibitor, a mast cell inhibitor, an MMP inhibitor, an NO inhibitor, an antibiotics, a vitamin, a histone deacetylase inhibitor, an anesthetics, sucralfate, and an inhibitor of NF-kappaB.
